(19) United States
(12) Patent Application Publication
(10) Pub. No.: US 2026/0057518 A1
(43) Pub. Date: Feb. 26, 2026

(54) TUMOR PREDICTION SYSTEM AND METHOD BASED ON TONGUE IMAGE AND TUMOR MARKER, AND APPLICATION THEREOF

(71) Applicant: ZHEJIANG CANCER HOSPITAL, Hangzhou City, Zhejiang Province (CN)

(72) Inventors: XIANGDONG CHENG, Hangzhou City, Zhejiang Province (CN); LI YUAN, Hangzhou City, Zhejiang Province (CN); LIN YANG, Hangzhou City, Zhejiang Province (CN); SHICHUAN ZHANG, Hangzhou City, Zhejiang Province (CN); ZHIYUAN XU, Hangzhou City, Zhejiang Province (CN)

(21) Appl. No.: 18/997,512

(22) PCT Filed: Jun. 29, 2023
(86) PCT No.: PCT/CN2023/103812

§ 371 Date: Apr. 30, 2025

(30) Foreign Application Priority Data
Jul. 22, 2022 (CN) 202210860206.6

(51) Int. Cl. G06T 7/00; A61B 5/00; G06V 10/50; G06V 10/764

(52) U.S. Cl. CPC G06T 7/0014; A61B 5/4552; G06V 10/50; G06V 10/764; G06T 2207/20021; G06T 2207/20081; G06T 2207/30096; G06T 2207/30204; G06V 2201/03

(57) ABSTRACT

The invention pertains to oncology diagnosis, prediction, and evaluation, specifically a tumor prediction system and method utilizing tongue images and hematological tumor markers, and the application thereof. The system includes: a tongue image acquisition module for capturing tongue images; a hematological tumor marker acquisition module for obtaining tumor marker indices; and a data processing module that predicts the probability of a test sample being positive for tumors using an AI deep learning model. This model analyzes discriminative features from both tongue images and hematological data to make joint decisions, offering a prospective, economical, non-invasive, and effective screening and diagnostic tool for tumors.

### TECHNICAL FIELD

The invention relates to the technical field of oncology diagnosis, prediction and evaluation, in particular to a tumor prediction system, a method and application thereof based on a tongue picture image and a blood tumor marker, which realize economical and non-invasive tumor prediction with higher accuracy by analyzing the correlation between the tongue picture image and the blood tumor marker and oncology.

### BACKGROUND ART

According to the latest data, gastric cancer (GC) is the third largest cause of cancer-related deaths in the world, with 1.09 million new GC cases and 770,000 deaths in 2020 alone, including 480,000 new cases and 370,000 deaths in China, accounting for about half of the world's cases. China is a country with high incidence and mortality of gastric cancer. Early detection, early diagnosis and early treatment are the key to reduce the mortality of gastric cancer. However, the diagnosis rate of early gastric cancer in China is still less than 20%. Under the condition of large population base, gastroscopy screening is only aimed at the target population meeting specific conditions, but its application is greatly limited because of its strong invasiveness, high cost and the need for professional endoscopists. Studies have confirmed that the expression of carcinoembryonic antigen and glycoprotein antigen is closely related to the growth, staging, differentiation, invasion and lymph node metastasis of gastric cancer, and plays an important role in the progress of gastric cancer, so the detection of blood indicators is one of the main means of clinical application.

Unfortunately, in the early stage of gastric cancer, due to the lack of specific symptoms, the specificity and sensitivity of clinical disease markers are poor, and more than 60% of patients have local or distant metastasis at the time of diagnosis. The 5-year survival rate of patients with local early GC was more than 60%, while the 5-year survival rate of patients with local and distant metastases decreased significantly to 30% and 5%, respectively. Therefore, there is an urgent need for new diagnostic or screening methods for GC to improve the early diagnostic rate and prognostic effect in this population.

Traditional Chinese medicine (TCM) is a medical science and cultural heritage that has been applied and retained by the Chinese people for thousands of years. Tongue diagnosis is one of the important bases for TCM to diagnose diseases. According to the theory of traditional Chinese medicine, the change of tongue picture (the color, size and shape of the tongue, the color, thickness and water content of the tongue coating) can reflect the health status of the human body, especially closely related to stomach diseases. However, no study has confirmed that there is a corresponding relationship between tongue changes and GC, and the value of tongue changes in the diagnosis and screening of GC.

Artificial intelligence (AI) can be used to screen, diagnose and treat various diseases, and scholars such as CheungCY have developed a deep learning system (see references) to assess the risk of cardiovascular disease by measuring the caliber of retinal blood vessels, which can effectively predict the risk of cardiovascular disease. Takenaka K et al. developed a deep neural network (see references) to evaluate endoscopic images of patients with ulcerative colitis, which identified patients with endoscopic remission and histological remission with 90.1% accuracy and 92.9% accuracy.

The patent CN110251084A of Fuzhou Data Technology Research Institute Co., Ltd. provides a tongue image detection and recognition method based on artificial intelligence, which is used to solve the real-time detection, shooting, saving and uploading of tongue image and tongue body in the process of tongue image acquisition, and to recognize tongue image, tongue color, tongue shape, tongue coating and tongue coating color. The scheme mainly involves tongue image acquisition and recognition technology, in which tongue image recognition focuses more on the extraction of tongue image color, texture, tongue coating area or tongue coating thickness and other characteristics, but these works do not establish a corresponding relationship between tongue image information and a special stomach disease such as gastric cancer. The patent CN111710394A of Shenyang Zhilang Technology Co., Ltd. proposes an early gastric cancer screening system assisted by artificial intelligence, which solves the problem of heavy workload of gastric cancer positive determination by replacing manual analysis of gastroscope slice images with automation. However, this strategy based on gastroscope image analysis still needs to obtain a large number of gastroscope images collected by professional instruments for model learning, and still needs to make decisions according to the gastroscope images of each tester in the testing stage. However, the acquisition of gastroscope images still has the disadvantages of high time consumption, high material cost and high standard of testing population. It is difficult to achieve nationwide census screening.

Jiangsu Tianrui Precision Medical Technology Co., Ltd. CN112133427A provides an auxiliary diagnosis system for gastric cancer based on artificial intelligence, which comprises a diagnosis selection module, a data acquisition module, a preprocessing module, a diagnosis module and a display output module. The system can give a diagnosis result in a personalized manner according to the collected data of a patient. The diagnostic system is based on data including basic information, diet, infection history, disease history, family history, clinical signs and symptoms and test items, among which the collection of data such as clinical signs and symptoms and test items is more difficult, while the effect of early screening and diagnosis will be affected if only relying on basic information, diet, infection history, disease history, family history and other information.

### REFERENCES

Cheung C Y, Xu D, Cheng C Y, et al. A deep-learning system for the assessment of cardiovascular disease risk via the measurement of retinal-vessel calibre. Nature biomedical engineering 2021; 5 (6): 498-508. doi: 10.1038/s41551-020-00626-4 [published Online First: 2020 Oct. 14];
Takenaka K, Ohtsuka K, Fujii T, et al. Development and Validation of a Deep Neural Network for Accurate Evaluation of Endoscopic Images from Patients with Ulcerative Colitis. Gastroenterology 2020; 158 (8): 2150-57. doi: 10.1053/j.gastro.2020.02.012 [published Online First: 2020 Feb. 16].

The present invention seeks to address these and other outstanding needs in the art.

### SUMMARY OF THE INVENTION

In order to solve at least one of the technical problems mentioned in the above background technology, the purpose of the present invention is to provide a tumor prediction system based on tongue images and blood tumor markers, aiming at applying AI deep learning model to automatically predict the probability of different test samples belonging to tumor positive according to the joint decision of tongue images and clinical blood tumor markers. The tumor prediction system has the advantages of simple operation, low cost, painlessness and non-invasiveness, and a large number of test cases prove that the prediction system is a prospective, economical, non-invasive and effective screening and diagnosis prediction system for tumors.

The invention relates to a tumor prediction system based on a tongue image and a blood tumor marker, comprising:
A tongue image acquisition module configured to acquire a tongue image of a test specimen;
A blood tumor marker acquisition module configured to acquire a blood tumor marker index of the test sample;
A data processing module configured to obtain a probability that the test specimen is positive by:

Predicting the positive probability of the test sample according to the discriminable characteristics of the tongue image obtained by automatic learning and the blood tumor marker index data modality.

In a specific embodiment, the tongue image of the test sample obtained by the tongue image obtaining module may be obtained by at least one of photographing, network transmission, and import. The tongue picture image used for training in the system can be obtained in at least one way of pre-storage, network transmission, import, etc., and can be obtained and imported into the system in a conventional way.

In a specific embodiment, the tongue image is a complete tongue image of the sample, and the tongue region is clearly distinguished from the background region.

In a specific embodiment, the blood tumor marker acquisition module can obtain the blood tumor marker indicators of the test sample through at least one method such as network transmission, import, local storage, testing, etc. The blood tumor marker acquisition module aims to obtain the blood tumor marker indicators of the sample. The blood tumor marker indicators used for training within the system can be obtained through at least one method such as pre-storage, network transmission, import, etc. They can be obtained and imported into the system in a conventional manner.

In a specific embodiment, The blood tumor marker is selected from the group consisting of alpha-fetoprotein (AFP), carcinoembryonic antigen (CEA), cancer antigen 125 (CA125), cancer antigen 15-3 (CA15-3), cancer antigen 199 (CA199), cancer antigen 72-4 (CA72-4), cancer antigen 242 (CA242), cancer antigen 50 (CA50), Non-small cell lung cancer associated antigen (CYFRA21-1), small cell lung cancer associated antigen (neuron-specific enolase, NSE), squamous cell carcinoma antigen (SCC), total prostate specific antigen (TPSA), free prostate specific antigen (FPSA), alpha-L-fucosidase (AFU), Epstein-Barr virus antibody (EBV-VCA), tumor-related substance (TSGF), Ferritin, .beta.2-microglobulin (.beta.2-MG), pancreatic embryonic antigen (POA) or gastrin precursor releasing peptide (PROGRP), in particular at least one selected from CEA, CA242, CA72-4, CA125, CA199, CA50, AFP or Ferritin, More particularly, a combination of the above CEA, CA242, CA72-4, CA125, CA199, CA50, AFP and Ferritin is selected.

In a particular embodiment, the tumor is at least one of gastric cancer, breast cancer, colorectal cancer, esophageal cancer, hepatobiliary pancreatic cancer, lung cancer, prostate cancer, thyroid cancer, ovarian cancer, neuroblastoma, trophoblastic tumor, or head and neck squamous cell carcinoma.

In a specific embodiment, the tumor is at least one of gastric cancer, breast cancer, colorectal cancer, esophageal cancer, hepatobiliary pancreatic cancer, or lung cancer.

In a specific embodiment, the system further includes an output module configured to output the prediction result.

In a specific embodiment, the output module is configured to output the tongue picture image and the prediction result.

In a specific embodiment, the output module outputs in at least one mode of electronic display, audio broadcast, printing, and network transmission.

In a specific embodiment, the discriminative feature is derived from the positive category and the negative category on the tongue image and the blood tumor marker index data modality.

Aiming at obtaining the discriminability characteristics between the positive category and the negative category by fully comparing, analyzing and learning the commonness and difference among and between the positive tongue picture image, the blood tumor marker index and/or the negative tongue picture image and the blood tumor marker index, and judging the probability that the test sample belongs to the positive category by deeply judging the discriminability characteristics of the test sample, So that the tumor prediction of the test sample can be realized by combining the tongue image with the blood tumor marker index. The discriminability characteristics can come from the commonness and difference among the positive tongue image, the blood tumor marker index, the negative tongue image and the blood tumor marker index, and can also come from the commonness and difference among the positive category and the negative category on the single tongue image and the blood tumor marker index data modality, That is to say, the discriminability characteristics between the positive category and the negative category obtained from the tongue image and the blood tumor marker index data modality can be used to predict whether the test sample belongs to the positive category or the negative category.

The discriminability feature comes from the positive tongue image and the corresponding hematological tumor marker index and the negative tongue image and the corresponding hematological tumor marker index which are input into the interactive deep learning model in pairs.

In one embodiment, the data processing module is configured to predict a probability that the test specimen is positive by:

The positive tongue image, the corresponding blood tumor marker index, the negative tongue image and the corresponding blood tumor marker index which are input into the interactive deep learning model at the same time are fully compared, and the similarities and differences between the positive category and the negative category on the data mode of the tongue image and the blood tumor marker index are automatically learned. The probability of the test sample being positive is predicted based on the characteristics of the discriminability between the positive and negative categories.

It should be clear that the present application aims to analyze, judge and predict the tumor positive and negative probability of an organism by analyzing and learning a tongue image and a blood tumor marker index from the organism, so the tongue image and the blood tumor marker index are both collected from the same sample, and further, are collected from the same organism, which is the meaning of the following corresponding expressions; Therefore, it is inappropriate to use tongue images collected from one body and blood tumor markers collected from another body as predictive sources.

The scheme aims to obtain the discriminability characteristics between the positive category and the negative category by fully comparing, analyzing and learning the commonness and difference between the positive tongue picture image and the corresponding blood tumor marker index and the negative tongue picture image and the corresponding blood tumor marker index, and predict the probability that the test sample input into the model belongs to the positive category according to the discriminability characteristics, Therefore, any model that can compare, analyze and learn the commonness and difference between the positive tongue image and the corresponding blood tumor marker index and the negative tongue image and the corresponding blood tumor marker index, and then can obtain the discriminability characteristics between the positive category and the negative category, can be applied to the scheme of this part, and is also included in the scope of protection of the scheme of this part. In particular, the present application selects but is not limited to the APINet model combined with blood tumor markers for example analysis and illustration.

In a specific embodiment, the positive tongue image and the corresponding blood tumor marker index are collected from a tumor positive patient.

In a specific embodiment, the negative tongue image and the corresponding blood tumor marker index are collected from a tumor-negative patient.

In a specific embodiment, the interactive deep learning model is an APINet model.

In one embodiment, the data processing module is configured to obtain a probability that the test specimen is positive by:
1) extracting a positive feature and a negative feature from a pair of pre-acquired tongue images and a pair of pre-acquired blood tumor marker indexes;
2) training a model according to the positive characteristics and the negative characteristics, and outputting the probability that the characteristics belong to each category;
3) inputting the tongue image of the test sample and the blood tumor marker index into the trained model, and outputting the probability that the test sample is positive.

In a specific embodiment, the step of extracting the positive features and the negative features in step 1) comprises:

A coder extracts a characteristic vector of a tongue picture image, carries out splicing with a blood tumor marker index, carries out fusion through MLP of a fusion area, and outputs a positive characteristic f₁ and a negative characteristic f₂ after fusion;
Simultaneously inputting f₁, f₂ and the spliced feature fₘ into the MLP of the feature selection area, and correspondingly outputting two control vectors g₁ and g₂ corresponding to f₁ and f₂ respectively;
The activation of f₁ and f₂ by g₁ forms selected features f₁⁺ and f₂⁻, respectively, the activation of f₁ and f₂ by g₂ forms selected features f₁⁻ and f₂⁺, respectively. Two positive features f₁⁺ and f₁⁻ and two negative features f₂⁺ and f₂ are obtained.

In a specific embodiment, the MLP of the feature selection area fully learns the commonalities and differences of f₁ and fₘ and outputs the control vector g₁, and likewise learns the commonalities and differences of f₂ and fₘ and outputs the control vector g₂.

In a specific embodiment, the step 2) of training the model with the positive features and the negative features is specifically to input the positive features and the negative features into the fully connected layer classifier, and output the probabilities that these features respectively belong to each category.

In a specific embodiment, when the probability that the feature belongs to each category is output in step 2), the cross entropy loss function is minimized according to the categories of the four features:

$${L}_{C E} = - y log ( {φ}_{c} ( {f}_{i}^{k} ) ) , i ∈ \{ 1 , 2 \} , k ∈ \{ + , - \}$$

Where y is the true label corresponding to the feature, the function φ_{c} represents the final fully connected layer classifier, and fᵢ^{k} corresponds to the four input features.

Note that f₁⁺ is activated by the control vector g₁ corresponding to the positive feature and therefore contains positive feature information, while f₁⁻ is activated by the control vector g₂ corresponding to the negative feature and therefore contains negative feature information. The same applies to f₂⁺ and f₂⁻.

In a specific embodiment, when outputting the probability that the feature belongs to each category in step 2), considering that the confidence level output by the model for the feature fᵢ⁺ should be higher than that of the feature fᵢ⁻, the ranking loss function is minimized:

$${L}_{R} = max ( 0 , {p}_{i}^{+} ( c ) - {p}_{i}^{-} ( c ) + ϵ ) , i ∈ \{ 1 , 2 \}$$

Where, pᵢ⁻ and pᵢ⁺ it are the probability distributions of the features fᵢ⁻ and fᵢ⁻ on each category output by the classifier, ϵ∈[0, 1] is the specified hyper-parameter, and p (c) refers to the probability on the specified category c.

In a specific embodiment, inputting the tongue image and the blood tumor marker index of the test sample into the trained model in step 3) means inputting the tongue image and the blood tumor marker index of a single test sample.

In a specific embodiment, the probability of the output test sample belonging to the class in step 3) refers to the probability distribution of the test sample corresponding to the final output on each class, and the class corresponding to the maximum probability is taken as the predicted class.

In a specific embodiment, only the circumscribed rectangular part of the tongue surface area in the tongue image is used for training and testing, and the influence of the image background on the model can be effectively eliminated.

In a specific embodiment, in the training process, in order to enrich the sample space of the training set, the samples in the training set are randomly flipped at a certain probability, then the sub-images are cut at random positions on the image, and finally the images of fixed size are linearly interpolated and input into the interactive deep learning model after standardization.

High-quality sample data is a prerequisite for obtaining a high-generalization depth model, so positive and negative tongue images and corresponding blood tumor marker index data are obtained in advance from tumor patients and non-tumor people respectively. Only by fully comparing two pairs of samples (including a positive tongue image and its blood tumor markers and a negative tongue image and its blood tumor markers), can their similarities and differences be found, and the paired images and blood tumor markers are used as input to simulate the real scene. An encoder extracts image feature vectors, then splices the image feature vectors with blood tumor marker indexes, outputs positive features and negative features, combines the spliced features, finally outputs a pair of positive features and a pair of negative features, and inputs the positive features and the negative features into a fully-connected layer classifier to output the probability that the features respectively belong to each category, The cross-entropy loss function and the ranking loss function are simultaneously minimized to achieve the purpose of training the model. During the test, the tongue image of the test sample and its blood tumor marker index are input into the system to obtain the probability that it belongs to the positive tumor, and through in-depth analysis of the difference between the positive and negative tongue image and blood tumor marker index, the internal relationship between the tumor and tongue image information and blood tumor marker is learned based on in-depth learning technology. In view of the low accuracy of early cancer screening and the high cost of diagnostic strategies, the probability of positive cancer is automatically judged to screen out the high-risk population of cancer.

The aforementioned discriminability feature comes from the fact that:
A single positive tongue image and a corresponding blood tumor marker index, or
Single negative tongue image and corresponding blood tumor markers.

In a specific embodiment, the discriminative feature comes from the fact that the tongue image is cut into n small blocks and forms an input vector with the blood tumor marker index, and feature extraction is performed to obtain a deep feature that is beneficial to classification.

In one embodiment, the data processing module is configured to obtain a probability that the test specimen is positive by:

A tongue picture image of a t sample is cut into small blocks to form an input sequence, a blood tumor mark index is placed at that end of the input sequence to form an input vector, a position index is added to the input vector, the input vector is led into a trained deep learn model to carry out feature extraction and feature fusion, selected deep features beneficial to classification are output, and the probability of belonging to each category is obtained.

In a specific embodiment, the deep learning model is trained by the following steps:
a) cutting a tongue surface image into n small blocks, forming an input sequence according to the sequence, then placing a blood tumor marker index at the end of the input sequence to form an input sequence with the length of n+1, forming an input vector through linear mapping, and adding position indexes 0, 1, 2, . . . , n−1;
b) carrying out dimension amplification on the input blood tumor marker index through a full connection layer, aligning the input tumor marker index with an input vector mapped by a tongue surface image block, and endowing a position index n;
c) performing feature extraction and feature fusion by using an encoder based on the Transformer model, outputting the selected deep features which are beneficial to classification, and finally outputting the probability distribution of each category to which the deep features belong through the softmax classifier.

In a specific embodiment, the cutting of the tongue surface image into n small blocks in the aforementioned step a) means that the tongue picture image is cut into n square regions that do not overlap with each other.

In a specific embodiment, when the encoder performs feature extraction in step c), it includes L+1 Transformer layers, and each layer includes a self-attention mechanism.

In a specific embodiment, when the encoder performs feature extraction and feature fusion in step c), in order to remove redundant features, before the depth features are input to the last layer, a feature selection module comprising a multi-head attention mechanism performs region selection, and the feature selection module returns the index of the front row feature with the largest attention weight. The selected front row features are input to the last Transformer layer for feature fusion.

In one embodiment, the front row features are the first k features, k being one of 1, 2, 3, . . . , 20.

In one embodiment, the aforementioned k=12.

In a specific embodiment, the cross entropy loss function is minimized when the probability distribution of the output deep-seated features belonging to each class in the step c):

$${L}_{C E} = - \sum {y}_{i} log ( \tilde{{y}_{i}} ) , i ∈ \{ 0 , 1 \}$$

Where yᵢ is the element in the real one-hot annotation corresponding to the test sample. \tilde{y}ₗ is the probability that the model predicts to be the class yᵢ. The one-hot annotation is an annotation in the form of 0 and 1 vectors. For example, there are three categories. The one-hot annotations corresponding to the categories 0, 1 and 2 are (1, 0, 0), (0, 1, 0) and (0, 0, 1).

In a specific embodiment, when the probability distribution of the output deep feature belonging to each class in the step c) is obtained, the comparison loss function is minimized:

$${L}_{C} = \frac{1}{{N}^{2}} {\sum }_{i}^{N} [ {\sum }_{j : {y}_{i} = {y}_{j}}^{N} ( 1 - D ( {f}_{i} , {f}_{j} ) ) + {\sum }_{j : {y}_{i} \neq {y}_{j}}^{N} D ( {f}_{i} , {f}_{j} ) ]$$

Where N represents the size of the batch at the time of training, and the function D represents the similarity measure of the features fᵢ and fⱼ. In a training batch, all the negative and positive data pairs are selected to minimize the contrast loss, which makes the intra-class features more aggregated and the inter-class features more different, thus improving the prediction accuracy.

In the scheme of the part, a tongue picture image is cut into small areas which are not overlapped with each other, an input vector is formed by linear mapping after a sequence is formed in sequence, and then a blood tumor marker index is subjected to dimensional amplification through a fully connected layer, is aligned with the input vector mapped by a tongue surface image block, and is placed at the end of the input vector. Input the input vector into the TransFG model for feature extraction and feature fusion, generate deep features that are conducive to classification, and output the probability that they belong to each category through the softmax classifier, so as to complete the prediction of the category that the sample belongs to, and automatically predict and screen the tumor positive probability of the test sample through the automatic learning mode of the deep learning model. Compared with the problems of low accuracy of traditional early cancer screening and high cost of diagnostic strategies, this part of the program is based on tongue images and blood tumor markers, based on in-depth learning technology, to automatically determine the probability of tumor positive, in order to screen out the high-risk population of cancer. This part of the program is simple to operate, low cost and high test accuracy.

The tumor prediction method based on the tongue image and the blood tumor marker comprises the following steps:
Tongue images and blood tumor marker indexes of the test sample are obtained;
And inputting the tongue image and the blood tumor marker index of the test sample into the system to obtain the tumor positive probability of the test sample.

The application of the tumor prediction system and/or method based on the tongue image and the blood tumor marker comprises:

Tumor prediction is performed on a test sample using the system and/or method.

On the basis of general knowledge in the art, the above preferred conditions can be combined to obtain the specific embodiment.

The invention has the advantages that:

The invention provides a plurality of tumor prediction systems based on tongue images and blood tumor markers, which take non-organism sample tongue images and blood tumor marker indexes as direct implementation objects, can play an excellent diagnosis and prediction function for a plurality of tumors by analyzing and learning commonness and difference between positive characteristics and negative characteristics in the tongue images and combining the blood tumor marker indexes, Through the analysis and verification of a large number of real patient samples, the accuracy of the test for predicting gastric cancer can reach 75-81%, the sensitivity is 0.775-0.812, the specificity is 0.808-0.836, the accuracy is 0.810-0.866, and the AUC is 0.875-0.883; In external validation, the sensitivity was 0.858-0.866, the accuracy was 0.747-0.768, and the AUC was 0.834-0.835. The sensitivity and accuracy of the test were significantly better than those of the machine learning model based on blood tumor markers alone, and the accuracy was better than that of the tumor prediction system based on tongue images alone. The AUC value of the tumor prediction system based on tongue image and blood tumor markers was significantly higher than that of the tumor prediction system based on tongue image alone. Provides a prospective, economical, non-invasive and effective screening and diagnosis prediction system and method for tumors.

The invention adopts the technical scheme to achieve the purpose, overcomes the defects of the prior art, and has the advantages of reasonable design and convenient operation.

### BRIEF DESCRIPTION OF THE DRAWINGS

The foregoing and/or other objects, features, advantages and examples are set forth in the accompanying drawings, some of which are intended to be readily apparent to those skilled in the art. It should be noted that the drawings, illustrative embodiments and the description thereof, which are included to provide a further understanding of the present application, are not intended to unduly limit the present application.

FIG. 1 is a schematic diagram of a multi-center clinical study and its patient distribution;

FIG. 2 is a joint discrimination framework of the APINet fusion model;

FIG. 3 is a classification basis visualization of the APINet fusion model;

FIG. 4 is a schematic diagram of external validation and internal validation of three models based on blood tumor markers;

FIG. 5 is a schematic diagram of internal verification and external verification of a fusion model based on a tongue image and a blood tumor marker;

FIG. 6 is a joint discrimination framework of the TransFG fusion model;

FIG. 7 is a visualization of the results of the region selection module of the TransFG fusion model.

### DETAILED DESCRIPTION OF THE INVENTION

Appropriate substitution and/or modifications of that process parameters may be effected by those skilled in the art in light of the disclosure herein, however, it is specifically note that all such substitutions and/or modifications as would be apparent to one skilled in the art are deemed to be included herein. While the teachings of the present invention have been described with reference to the preferred embodiments, it will be apparent to those of ordinary skill in the art that the teachings of the present invention can be practiced and utilized with modification or appropriate alteration and combination of the teachings described herein without departing from the spirit and scope of the present invention.

It is noted that the following detailed description is exemplary in nature and is intended to provide further explanation of the present application. Unless otherwise indicated, all technical and scientific terms used herein have the same meaning as commonly understood by one of ordinary skill in the art to which this application belongs.

It should be noted that the terminology used herein is for the purpose of describing particular embodiments only and is not intended to be limiting of the application. As used herein, the singular forms are intended to include the plural forms as well, unless the context clearly dictates otherwise. It should also be understood that the terms “comprises” and/or “comprising,” when used in this specification, specify the presence of features, steps, operations, elements, components, and/or groups thereof.

APINet model: APINet model is the attentive pairwise interaction neural network (APINet) model.

TransFG model: TransFGmodel is the ransformer architecture for fine grained recognition (TransFG) model.

Hereinafter, the present invention is described in detail.

### <Clinical Specimen>

A nationwide multi-center clinical study was conducted to eliminate the influence of regional, dietary and center differences on the study, including 11 centers in 8 cities, located in Hangzhou, Wenzhou and Shanghai in the east, Fuzhou in the south, Chengdu in the west, Liaoning and Heilongjiang in the north, and Taiyuan in the central region.

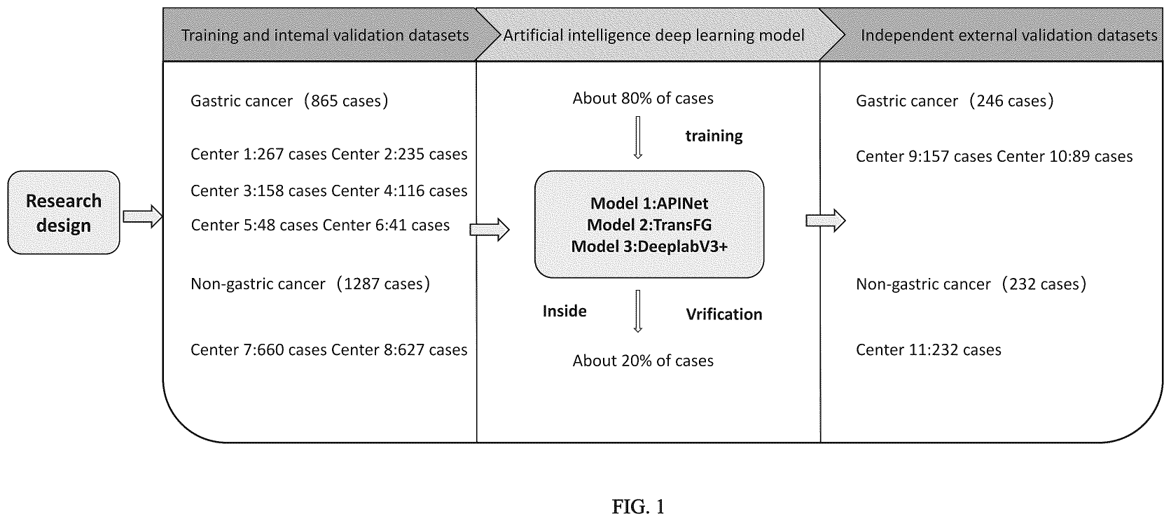

As shown in FIG. 1, from January 2020 to October 2021, 1111 gastric cancer (GC) patients and 1519 non-GC (NGC) patients were recruited from 8 centers, including 169 healthy controls (HCs), 648 superficial gastritis (SGs), and 702 atrophic gastritis (AGs). 865 patients with gastric cancer (GC) and 1287 patients without gastric cancer (NGC) were randomly selected to train and verify the system, including 448 patients with early GC (TNMI+II). 417 cases of advanced GC (TNMIII+IV), 141 cases of healthy controls (HC), 547 cases of superficial gastritis (SG), and 599 cases of atrophic gastritis (AG); approximately 80% of the cases were used as the training dataset, and approximately 20% of the cases were used as the internal validation dataset. In addition, 246 GCs and 232 NGCs from 3 centers were used as independent external validation datasets, including 162 early GCs, 84 late GCs, 28 HCs, 101 SGs, and 103 AGs. These gastric cancer (GC) patients were newly diagnosed with gastric cancer and had not received prior treatment for their disease, nor had they been treated with surgery, chemotherapy, radiotherapy, targeted therapy, or biotherapy for their disease. None of the gastric cancer (GC) patients had a single tumor, i.e., patients found to have two or more malignancies were also excluded. HCs, SGs and AGs were confirmed by gastroscopy.

Tongue images and clinical information of all participants were collected, including age, sex, height, weight, family history, smoking, drinking, TNM stage, blood tumor markers and so on. Pathological staging was based on the American Joint Committee on Cancer, 8th Edition, Issue 23. The tongue images of all GC participants were acquired on the morning of gastric surgery, and those of NGC participants were acquired on the morning of gastroscopy with an empty stomach for more than 8 hours, which excluded the influence of diet on tongue images. General patient information, such as age, gender, BMI, smoking, and alcohol consumption, between the GC and NGC groups are shown in Table 1 to be very well matched, whether in the training, internal validation, or independent external validation data sets.

TABLE 1
Clinical information for GC and NGC participants

| | [symbol] | | | [symbol] | | |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| | [symbol] | [symbol] | | [symbol] | [symbol] | |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

[symbol]

indicates data missing or illegible when filed

In addition, 104 patients with esophageal cancer (EC), 129 patients with hepatobiliary pancreatic cancer (HBPC), 116 patients with colorectal cancer (CRC), 260 patients with lung cancer (LC), and 154 patients with breast cancer (BC) were recruited from Zhejiang Cancer Hospital. Table 2 shows the clinical information of the other cancer participants, and it can be seen that the general information between GC and other cancers, such as age, sex, BMI, smoking and alcohol consumption, is well matched, except for BC.

TABLE 2
Clinical information of other cancer participants

| | | | [symbol] | | [symbol] | | [symbol] | | [symbol] | | [symbol] |
|---|---|---|---|---|---|---|---|---|---|---|---|
| [symbol] | GC | EC | [symbol] | [symbol] | [symbol] | CRC | [symbol] | LC | [symbol] | [symbol] | [symbol] |
| [symbol] | [symbol] | [symbol] | [symbol] | [symbol] | [symbol] | [symbol] | [symbol] | [symbol] | [symbol] | [symbol] | 0.113 |
| [symbol] | [symbol] | [symbol] | [symbol] | [symbol] | [symbol] | [symbol] | [symbol] | [symbol] | [symbol] | [symbol] | 0.000 |
| [symbol] | [symbol] | [symbol] | [symbol] | [symbol] | [symbol] | [symbol] | [symbol] | [symbol] | [symbol] | [symbol] | 0.922 |
| [symbol] | | | | | | | | | | | |
| [symbol] | [symbol] | [symbol] | [symbol] | [symbol] | [symbol] | [symbol] | [symbol] | [symbol] | [symbol] | [symbol] | 0.000 |
| [symbol] | [symbol] | [symbol] | [symbol] | [symbol] | [symbol] | [symbol] | [symbol] | [symbol] | [symbol] | [symbol] | 0.000 |
| [symbol] | [symbol] | [symbol] | [symbol] | [symbol] | [symbol] | [symbol] | [symbol] | [symbol] | [symbol] | [symbol] | 0.105 |
| [symbol] | | | | | | | | | | | |

[symbol]

indicates data missing or illegible when filed

### <Statistical Analysis>

All statistical analyses were performed using SPSS 23.0 software (SPSS Inc., Chicago, IL, USA). Results are expressed as mean±SD or mean±SEM. Parametric or nonparametric tests were used depending on whether the data were orthogonally distributed. Count data were analyzed using the chi-square test. P<0.05 was considered statistically significant.

### <Clinical Validation>

### Example 1

The joint validation of APINet model and blood tumor marker indicators is a tumor prediction system based on tongue image and blood tumor markers, referred to as APINet fusion model, which includes:
A tongue image acquisition module configured to acquire a tongue image of a test specimen;
A blood tumor marker acquisition module configured to acquire a blood tumor marker index of the test sample;
A data processing module configured to obtain a probability that the test specimen is positive by:
Predicting the positive probability of the test sample according to the discriminable characteristics of the tongue image obtained by automatic learning and the blood tumor marker index data modality.

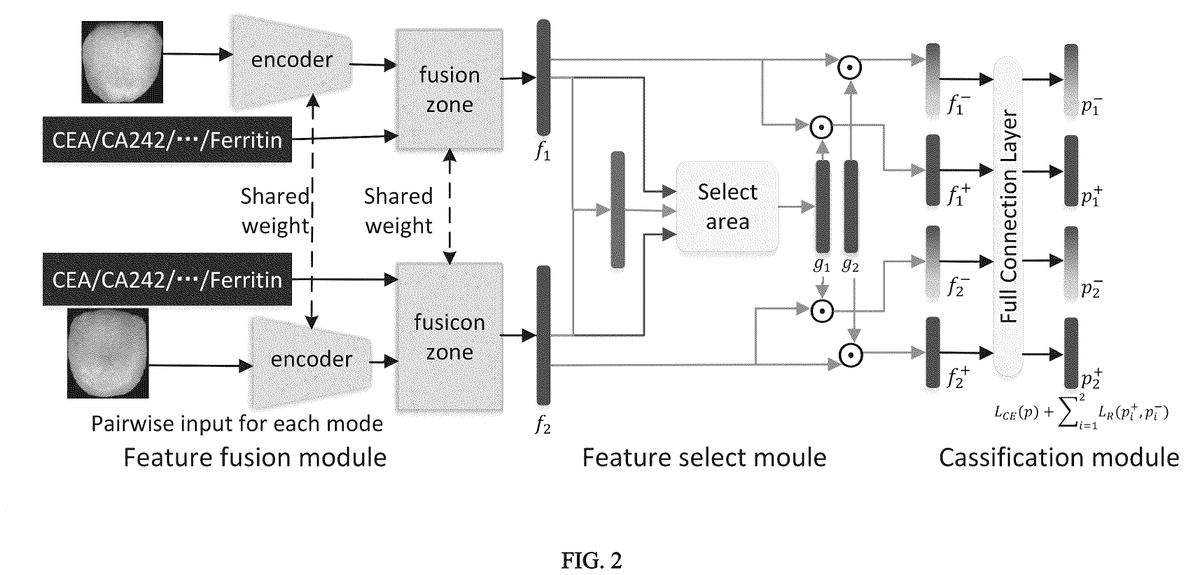

An interactive deep learning model based on comparison, APINet fusion model, is designed to automatically learn the similarities and differences between positive and negative categories in two data modalities by fully comparing a pair of tongue images and a pair of blood indicators that are in and out at the same time, and finally predict the probability that the test sample is tumor-positive according to the discriminative features. As shown in FIG. 2, the overall discrimination framework is divided into three modules: feature fusion module, feature selection module and classification module.

And that feature fusion module is use for simultaneously inputting a pair of tongue surface image which respectively belong to a positive category and a negative category, and correspondingly inputting a pair of blood tumor marker indexes. Firstly, the encoder extracts the feature vector of the image, directly splices with the blood index data, fuses through the MLP of the fusion area, and outputs the fused positive feature f₁ and negative feature f₂.

Feature selection module: simultaneously input f₁, f₂ and the spliced feature fₘ into the MLP of the feature selection area, and correspondingly output two control vectors g₁ and g₂, respectively corresponding to f₁ and f₂. Activating f₁ and f₂ respectively with the control vector g₁ to form selected features f₁⁺ and f₂⁻, activating f₁ and f₂ respectively with the control vector g₂ to form selected features f₁⁻ and f₂⁺, Two positive features f₁⁺ and f₁⁻ and two negative features f₂⁺ and f₂⁻ are obtained.

Classification module: We input the selected features to the fully connected layer classifier, and finally output the probability that these features belong to each category. In the training process, the cross entropy loss function is minimized according to the categories to which the four features belong:

$${L}_{C E} = - y log ( {φ}_{c} ( {f}_{i}^{k} ) ) , i ∈ \{ 1 , 2 \} , k ∈ \{ + , - \}$$

Where y is the true label corresponding to the feature, the function φ_{c} represents the final fully connected layer classifier, and fᵢₖ corresponds to the four input features. A model with better generalization should output higher confidence for feature fᵢ⁺ than for feature fᵢ⁻, so we minimize a ranking loss function at the same time:

$${L}_{R} = max ( 0 , {p}_{i}^{+} ( c ) - {p}_{i}^{-} ( c ) + ϵ ) , i ∈ \{ 1 , 2 \}$$

Where, pᵢ⁻ and pᵢ⁺ are the probability distributions of the features fᵢ⁻ and fᵢ⁺ on each category output by the classifier, ϵ∈[0, 1] is the specified hyper-parameter, and p (c) refers to the probability on the specified category c.

When the model is tested, only the feature fusion module and the classification module are reserved, the paired input of positive and negative data during training is changed into the input of a single test sample (including a tongue image and a corresponding blood tumor marker index), the probability distribution of the output of the corresponding test sample on each category is finally output, and the category corresponding to the maximum probability is taken as the predicted category.

A total of 905 related patients were tested, including 427 internal tests from the same center as the training set, and 478 external tests from different centers. The test results are shown in Table 3 and Table 4 below.

TABLE 3
Internal Test Results

Pediction category

| | | |
|---|---|---|
| internal testing | Negative | Positive |

| | | | |
|---|---|---|---|
| actual category | Negative | 173 | 41 |
| | Positive | 40 | 173 |

TABLE 4
External Test Results

Pediction category

| | | |
|---|---|---|
| internal testing | Negative | Positive |

| | | | |
|---|---|---|---|
| actual category | Negative | 146 | 86 |
| | Positive | 35 | 211 |

In Table 3, the actual number of negative cases is 173+41=214 cases, and the actual number of positive cases is 40+173=213 cases; the prediction results show that among the negative cases, 173 cases are correctly predicted to be negative, and 41 cases are incorrectly predicted to be positive; among the positive cases, 173 cases are correctly predicted to be positive, and 40 cases are incorrectly predicted to be negative; Therefore, the prediction accuracy in the internal test is (the number of negative predictions+the number of positive predictions)/the total number of test samples=(173+173)/(173+41+40+173)=81%. Similarly, it can be seen from Table 4 that the accuracy of the external test can reach 75%. From the results of internal and external tests, it can be seen that this part of the tumor diagnosis system has a good prediction accuracy for gastric cancer.

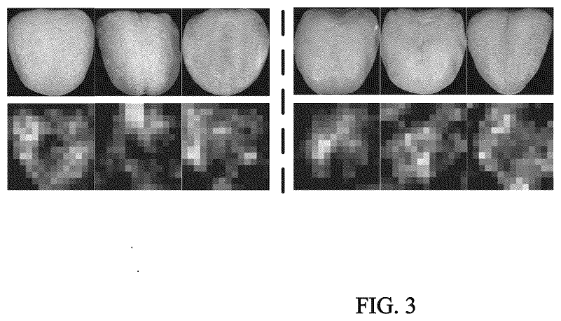

FIG. 3 shows the visualization of the model classification basis. The three test samples in the first row on the left side of the dotted line are positive tongue surface images, the second row is the area on which the model is mainly based for recognition based on the tongue surface images, and the right side of the dotted line is the negative sample and the corresponding visualized image of the tongue surface recognition basis. In the second row of images, the darker the color is, the more attention the model pays to the region. From the results shown, it is found that the region on which the model recognition process is based is mainly concentrated on the tongue surface, and has nothing to do with the black background.

In order to further evaluate the value of tongue images combined with blood tumor markers as a means of diagnosing and screening tumors, we compared the prediction system based on tongue images and blood tumor markers with the model based solely on blood tumor markers with clinical value.

As a contrast, the combination of a variety of classical blood tumor markers was used to verify the prediction of tumors. The selectable hematological tumor marker is selected from the group consisting of alpha-fetoprotein (AFP), carcinoembryonic antigen (CEA), cancer antigen 125 (CA125), cancer antigen 15-3 (CA15-3), cancer antigen 199 (CA199), cancer antigen 72-4 (CA72-4), cancer antigen 242 (CA242), cancer antigen 50 (CA50), Non-small cell lung cancer associated antigen (CYFRA21-1), small cell lung cancer associated antigen (neuron-specific enolase, NSE), squamous cell carcinoma antigen (SCC), total prostate specific antigen (TPSA), free prostate specific antigen (FPSA), alpha-L-fucosidase (AFU), Epstein-Barr virus antibody (EBV-VCA), tumor-related substance (TSGF), Ferritin, .beta.2-microglobulin (.beta.2-MG), pancreatic embryonic antigen (POA) or gastrin precursor releasing peptide (PROGRP), in particular at least one selected from CEA, CA242, CA72-4, CA125, CA199, CA50, AFP or Ferritin, More particularly, the combination of the eight blood tumor markers is selected.

The prediction method based on the blood tumor marker index comprises the following steps:
1) Data preprocessing: Because the serum indicators of all cases are missing to varying degrees, the training data need to be complete. Therefore, the data need to be completed before the model is trained, and the K neighbor missing value interpolation method is adopted to complete the data; Specifically, the missing serum index completion value is the average of the values of the two nearest neighbors;
2) model train: that invention adopts three machine learn classification methods, namely a support vector machine (SVM), a decision tree (DT) and a K-nearest neighbor classifier (KNN), The eight blood tumor markers (CEA, CA242, CA72-4, CA125, CA199, CA50, AFP and Ferritin) of the cases correspond to the sample characteristics, and the negative and positive diagnosis of the cases correspond to the sample labels. All the completed samples are sent to the three classifiers for fitting.
3) Model evaluation: the application adopts internal verification and external verification to evaluate the model; The internal validation uses the data of different cases in the same hospital as the training data, while the external validation uses the data of different hospital cases from the training data. Three indicators including sensitivity, specificity and accuracy were used to predict the model.

The clinical information of hematological tumor markers of related GC patients is shown in Table 5. Compared with NGC patients, the concentrations of hematological tumor markers such as CEA, CA424, CA724, CA125, CA199, CA50, AFP and Ferritin were significantly higher in GC patients.

TABLE 5
Clinical information of blood tumor markers in patients with GC

| | [symbol] | | | [symbol] | | |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | / |
| | [symbol] | [symbol] | | [symbol] | [symbol] | |
| | | [symbol] | | | [symbol] | |
| [symbol] | [symbol] | [symbol] | 0.000 | [symbol] | [symbol] | [symbol] |
| | [symbol] | [symbol] | | [symbol] | [symbol] | |
| [symbol] | [symbol] | [symbol] | 0.000 | [symbol] | [symbol] | [symbol] |
| | [symbol] | [symbol] | | [symbol] | [symbol] | |
| [symbol] | [symbol] | [symbol] | 0.000 | [symbol] | [symbol] | 0.000 |
| | [symbol] | [symbol] | | [symbol] | [symbol] | |
| [symbol] | [symbol] | [symbol] | 0.013 | [symbol] | [symbol] | 0.074 |
| | [symbol] | [symbol] | | [symbol] | [symbol] | |
| [symbol] | [symbol] | [symbol] | 0.000 | [symbol] | [symbol] | 0.022 |
| | [symbol] | [symbol] | | [symbol] | [symbol] | |
| [symbol] | [symbol] | [symbol] | 0.000 | [symbol] | [symbol] | 0.003 |
| | [symbol] | [symbol] | | [symbol] | [symbol] | |
| [symbol] | [symbol] | [symbol] | 0.001 | [symbol] | [symbol] | 0.185 |
| | [symbol] | [symbol] | | [symbol] | [symbol] | |
| [symbol] | [symbol] | [symbol] | 0.000 | [symbol] | [symbol] | / |
| | [symbol] | [symbol] | | | [symbol] | |

[symbol]

indicates data missing or illegible when filed

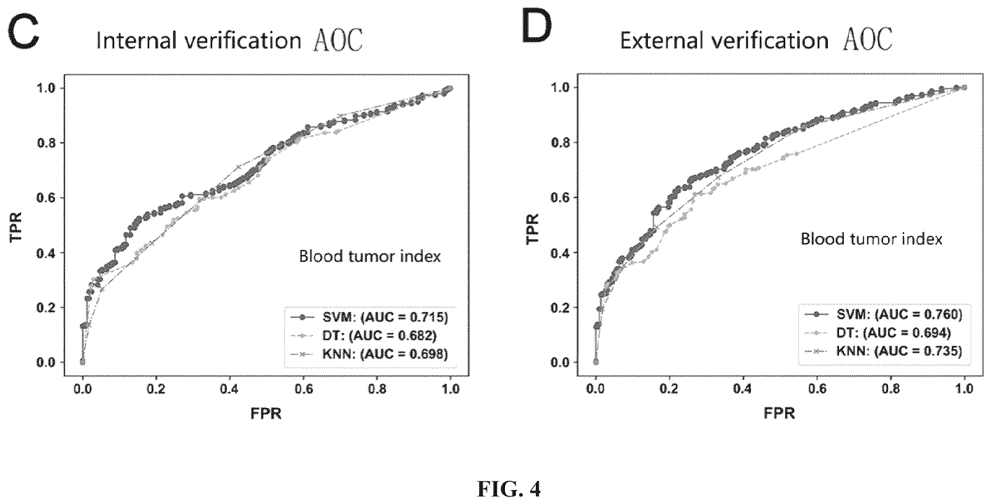

The training, internal validation and external validation data sets of the model are consistent with the model based on tongue images and blood tumor markers (excluding the absence of blood indicators). The sensitivity, specificity and accuracy verification results of the blood tumor markers based on the three machine learning classification methods for GC diagnosis are shown in Table 6, and the ROC and AUC for internal and external verification are shown in FIG. 4. The AUC values for internal verification range from 0.682 to 0.715. The AUC values of external validation ranged from 0.694 to 0.760. The specificity of internal validation and external validation reached more than 90% in SVM algorithm, indicating that the algorithm can provide valuable information for the diagnosis of gastric cancer. In DT and KNN, the specificity was decreased, the sensitivity and accuracy were improved in varying degrees, which can provide a variety of information for the diagnosis of gastric cancer.

TABLE 6
Sensitivity, Specificity and Accuracy of the Model
Based on Blood Tumor Markers for GC Diagnosis

| | [symbol] | | | [symbol] | | |
|---|---|---|---|---|---|---|
| | sex | specificity | accuracy | sex | specificity | accuracy |
| SVM | 0.283 | 0.976 | 0.603 | 0.362 | 0.938 | 0.645 |
| DT | 0.566 | 0.688 | 0.622 | 0.539 | 0.759 | 0.647 |
| KNN | 0.434 | 0.812 | 0.609 | 0.496 | 0.835 | 0.662 |

[symbol]

indicates data missing or illegible when filed

It should be clear that the above comparison scheme of the present application selects eight serum indicators including CEA, CA242, CA72-4, CA125, CA199, CA50, AFP and Ferritin, and the increase, reduction or replacement of several serum indicators can predict the negative and positive of tumors, especially gastric cancer. Three machine learning classifiers SVM, DT and KNN are used in the above comparison scheme, and other machine learning classifier methods such as logistic regression and random forest can also be used to achieve the corresponding purpose.

Compared with the SVM, DT, and KNN, the sensitivity, specificity, and accuracy of the APINet fusion model of this embodiment for GC diagnosis are improved or changed to varying degrees, as shown in Table 7.

TABLE 7
Sensitivity, Specificity and Accuracy of APINet Fusion Model for GC Diagnosis

| | [symbol] | | | [symbol] | | |
|---|---|---|---|---|---|---|
| | sex | specificity | accuracy | sex | specificity | accuracy |
| APINet fusion model | 0.812 | 0.808 | 0.810 | 0.858 | 0.629 | 0.747 |

[symbol]

indicates data missing or illegible when filed

Table 7 shows the sensitivity, specificity and accuracy data of the tongue image-based APINet fusion model for GC diagnosis. It can be seen that the APINet fusion model has significantly higher sensitivity to GC diagnosis in both internal and external validation than SVM, DT and KNN models based on eight blood tumor markers (0.812 and 0.283-0.566, 0.858 and 0.362-0.539) and accuracy (0.810 and 0.603-0.622, 0.747 and 0.645-0.662), especially the system based on tongue image and blood tumor markers showed high specificity (0.808) in internal verification. Provides a prospective, economical, non-invasive and effective screening and diagnosis prediction method for tumors.

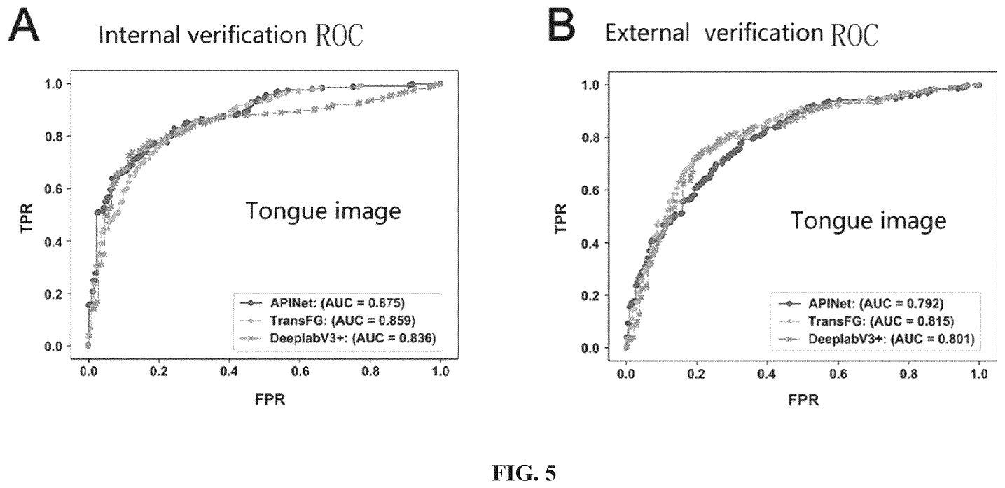

The APINet fusion model fully compares two pairs of input data (positive tongue image and corresponding blood tumor marker index of the test sample, negative tongue image and corresponding blood tumor marker index of the test sample) through pairwise interaction to identify contrast clues for classification. FIG. 5 shows the ROC (Receiver Operating Characteristic) and AUC (Area Under ROC Curve) of the internal validation (API_I in FIG. 5) and external validation (API_E in FIG. 5) of the APINet fusion model. Compared with the SVM, DT and KNN models in FIG. 4, the APINet fusion model in FIG. 5 has a ROC curve far away from the (0, 0)-(1, 1) line in both internal and external validation, with an internal validation AUC value of 0.875 and an external validation AUC value of 0.835. It was significantly higher than internal validation AUC values (0.682-0.715) and external validation AUC values (0.694-0.760) of SVM, DT and KNN models of eight blood tumor markers, and slightly higher than model without blood tumor markers and only using tongue images. It is concluded that the APINet fusion model is a good prediction model, and the tongue image combined with blood tumor markers can further improve the diagnostic value of tumors. The AI diagnostic model based on tongue image and blood tumor markers is superior to the model based on the combination of eight blood tumor markers and the model based on tongue image.

In addition, it can be known from the corresponding prediction results of tumors including breast cancer, colorectal cancer, esophageal cancer, hepatobiliary pancreatic cancer, lung cancer and the like by applying the model of the embodiment that a diagnosis prediction result with an AUC of not less than 0.500 can be obtained, and it can be known that the system provided by the present application can perform economical, non-invasive and effective screening and diagnosis prediction on the tumors.

### Example 2

A tumor prediction system based on tongue imaging and blood tumor markers, referred to as the TransFG fusion model, is jointly validated using the TransFG model and blood tumor marker indicators. It includes:
A tongue image acquisition module configured to acquire a tongue image of a test specimen;
A blood tumor marker acquisition module configured to acquire a blood tumor marker index of the test sample;
A data processing module configured to obtain a probability that the test specimen is positive by:
Predicting the positive probability of the test sample according to the discriminable characteristics of the tongue image obtained by automatic learning and the blood tumor marker index data modality.

A combined diagnosis model of tongue image and blood indicators based on deep learning, TransFG fusion model, is designed, which automatically predicts the probability of different test objects belonging to tumor positive according to tongue image and clinical blood tumor markers.

Annotated data with high quality is a prerequisite for obtaining a deep model with high generalization. Positive and negative tongue image data were obtained from gastric cancer patients and non-gastric cancer patients respectively, and eight clinical blood indexes: CEA, CA424, CA72-4, CA125, CA199, CA50, AFP and Ferritin were collected for each sample one by one. Based on the data of the above two modes (tongue image and blood tumor marker index), a deep learning model based on Transformer is designed, which divides the input tongue image into small blocks without overlap, and then inputs the separated small blocks into the deep neural network in sequence. Blood tumor marker indicators are placed at the end of the input sequence as auxiliary diagnostic data. Finally, the probability that the test sample is positive is predicted according to the extracted discriminative features.

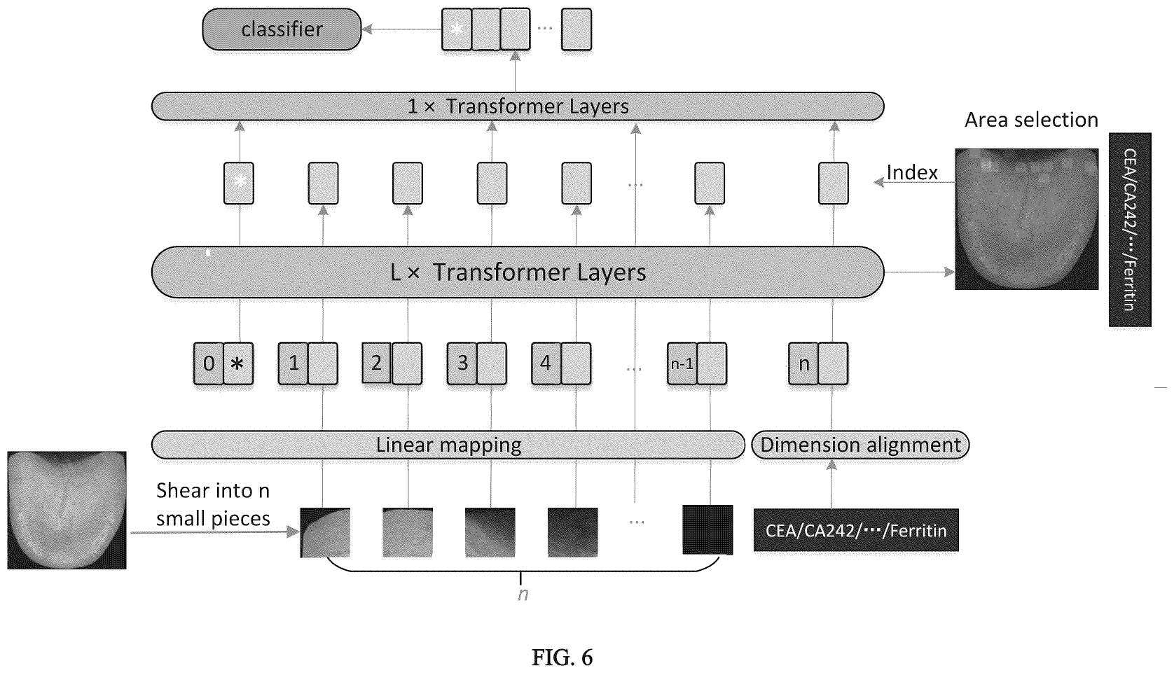

The joint discrimination framework based on tongue image and blood tumor markers is shown in FIG. 6. The input of the whole model is the tongue image and its corresponding eight clinical blood tumor markers.

Firstly, the tongue surface image is cut into n small blocks, and then the cut n small blocks form an input sequence in order. In order to use the blood tumor marker index as the basis for diagnosis and screening, we put eight blood tumor marker indexes at the end of the input sequence to form an input sequence with a length of n+1. Specifically, the small image blocks are linearly mapped to form an input vector, and position indexes 0, 1, 2, . . . , n−1; The input blood tumor marker index is dimensionally expanded through the fully connected layer, aligned with the input vector mapped by the image block, and endowed with a position index n.

The encoder based on the Transformer model is used for feature extraction, which includes L+1 transformer layers, and each layer contains a self-attention mechanism. In order to remove the redundant features, before the deep features are input to the last layer, a feature selection module is used for region selection, the module comprises a multi-head attention mechanism, the index of the k block features with the largest attention weight is returned, the k selected features are input to the Transformer layer of the last layer for feature fusion, The selected deep features that are beneficial to classification are output, and finally the probability distribution of each category is output through the softmax classifier.

The cross entropy loss function is minimized separately with the probability distribution of the output:

$${L}_{C E} = - \sum {y}_{i} log ( \tilde{{y}_{i}} ) , i ∈ \{ 0 , 1 \}$$

and minimize that contrast loss function:

$${L}_{C} = \frac{1}{{N}^{2}} {\sum }_{i}^{N} [ {\sum }_{j : {y}_{i} = {y}_{j}}^{N} ( 1 - D ( {f}_{i} , {f}_{j} ) ) + {\sum }_{j : {y}_{i} \neq {y}_{j}}^{N} D ( {f}_{i} , {f}_{j} ) ]$$

so that the intra-class features are more aggregated and the inter-class features are more different, thereby improving the prediction accuracy.

We have tested 905 cases, including 427 internal tests from the same center as the training set, and 478 external tests from different centers. The test results are shown in Table 8 and Table 9 below. It can be seen that the accuracy of internal tests and external tests can reach 81% and 77% respectively. From the results of internal and external tests, it can be seen that the tumor diagnosis system has good prediction accuracy for gastric cancer.

TABLE 8
Internal Test Results

Pediction category

| | | |
|---|---|---|
| internal testing | Negative | Positive |

| | | | |
|---|---|---|---|
| actual category | Negative | 179 | 35 |
| | Positive | 48 | 165 |

TABLE 9
External Test Results

Pediction category

| | | |
|---|---|---|
| external testing | Negative | Positive |

| | | | |
|---|---|---|---|
| actual category | Negative | 154 | 78 |
| | Positive | 33 | 213 |

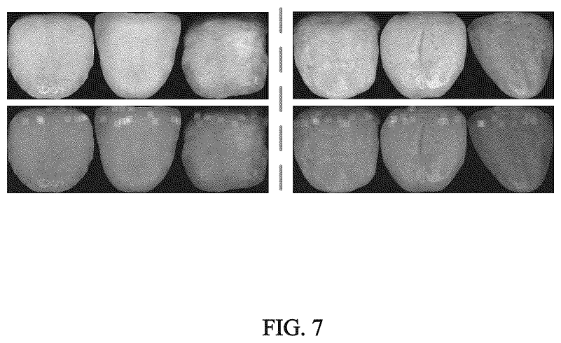

FIG. 7 shows the visualization of the result of the region selection module of the TransFG fusion model. The three test samples in the first row on the left side of the dotted line are positive tongue images, the small yellow blocks in the second row of images are the regions corresponding to the feature indexes returned by the region selection module in the original image, and the negative samples and the region selection result are on the right side of the dotted line. The results show that the regions on which the model recognition process is based are mainly concentrated in the upper part of the tongue with heavy tongue coating, and have low correlation with the black background and the lower part of the tongue.

In order to further evaluate the value of the combination of the tongue image and the blood tumor marker index as a means for diagnosing and screening tumors, we compared the prediction system based on the tongue image and the blood tumor marker described in this embodiment with the model based solely on the blood tumor marker with clinical value, and the latter is shown in Embodiment 1.

Compared with the SVM, DT, and KNN, the sensitivity, specificity, and accuracy of the TransFG fusion model in this embodiment for GC diagnosis are improved or changed to varying degrees, as shown in Table 10.

TABLE 10
Sensitivity, Specificity and Accuracy of TransFG Fusion Model for GC Diagnosis

| | [symbol] | | | [symbol] | | |
|---|---|---|---|---|---|---|
| | sex | specificity | accuracy | sex | specificity | accuracy |
| TransFG fusion model | 0.775 | 0.836 | 0.806 | 0.866 | 0.664 | 0.768 |

[symbol]

indicates data missing or illegible when filed

Table 10 shows the sensitivity, specificity and accuracy data of the TransFG fusion model based on tongue images for GC diagnosis. It can be seen that the sensitivity of TransFG fusion model in GC diagnosis in both internal and external validation is significantly higher than that of SVM, DT and KNN models based on eight blood tumor markers (0.775 and 0.283-0.566, 0.866 vs 0.362-0.539) and accuracy (0.806 vs 0.603-0.622, 0.768 vs 0.645-0.662), In particular, the system based on tongue images and blood tumor markers shows excellent specificity (0.836 and 0.688-0.976) in internal verification, and provides a prospective, economical, non-invasive and effective screening and diagnostic prediction method for tumors.

The TransFG fusion model sufficiently compares two pairs of input data (a positive tongue image and a corresponding blood tumor marker index of a test sample, and a negative tongue image and a corresponding blood tumor marker index of a test sample) through pairwise interaction to identify a contrast clue for classification. FIG. 5 shows the ROC and AUC of the internal validation (Trans_I in FIG. 5) and the external validation (Trans_E in FIG. 5) of the TransFG fusion model. The TransFG fusion model in FIG. 5 has a ROC curve farther away from the (0,0)-(1,1) line in both internal and external validation, with an internal validation AUC value of 0.883 and an external validation AUC value of 0.834. It was significantly higher than internal validation AUC values (0.682-0.715) and external validation AUC values (0.694-0.760) of SVM, DT and KNN models of eight blood tumor markers, and slightly higher than model without blood tumor markers and only using tongue images. TransFG fusion model is a good prediction model, and tongue images combined with blood tumor markers can further improve the diagnostic value of tumors. The AI diagnostic model based on tongue image and blood tumor markers is superior to the model based on the combination of eight blood tumor markers and the model based on tongue image.

In addition, it can be known from the corresponding prediction results of tumors including breast cancer, colorectal cancer, esophageal cancer, hepatobiliary pancreatic cancer, lung cancer and the like by applying the model of the embodiment that a diagnosis prediction result with an AUC of not less than 0.500 can be obtained, and it can be known that the system provided by the invention can perform economical, non-invasive and effective screening and diagnosis prediction on the tumors. It expands the existing means of cancer screening.

The conventional techniques in the above embodiments are known to those skilled in the art, and thus will not be described in detail herein.

The specific embodiments described herein are merely illustrative of the spirit of the invention. Those skilled in the art to which this invention pertains may make various modifications or additions to or substitutions in a similar manner for the specific embodiments described without departing from the spirit of the invention or going beyond the scope defined in the appended claims.

Although the present invention has been described in detail and illustrated with specific examples, it will be apparent to those skilled in the art that various changes and modifications can be made without departing from the spirit and scope of the invention.

The above description is only a preferred embodiment of the present application and is not intended to limit the present application, and various modifications and changes can be made to the present application by those skilled in the art. Any modification, equivalent substitution, improvement, etc. Within the spirit and principle of this application shall be included in the scope of protection of this application.

All matter not covered in that present invention are known in the art.

1. A tumor prediction system based on tongue images and blood tumor markers, comprising:
a tongue image acquisition module configured to acquire a tongue image of a test specimen;
a blood tumor marker acquisition module configured to acquire a blood tumor marker index of the test sample;
a data processing module configured to obtain a probability that the test specimen is positive by:
predicting the positive probability of the test sample according to the discriminable characteristics of the tongue image obtained by automatic learning and the blood tumor marker index data modality.

2. The system according to claim 1, wherein the discriminable feature is derived from a tongue image, between a positive category and a negative category on a hematological tumor marker index data modality.

3. The system of claim 2, wherein the data processing module is configured to predict a probability that the test sample is positive by:
the positive tongue image, the corresponding blood tumor marker index, the negative tongue image and the corresponding blood tumor marker index which are input into the interactive deep learning model at the same time are fully compared, and the similarities and differences between the positive category and the negative category on the data mode of the tongue image and the blood tumor marker index are automatically learned, the probability of the test sample being positive is predicted based on the characteristics of the discriminability between the positive and negative categories.

4. The system of claim 2, wherein the data processing module is configured to obtain a probability that the test sample is positive by:
1) extracting a positive feature and a negative feature from a pair of pre-acquired tongue images and a pair of pre-acquired blood tumor marker indexes;
2) training a model according to the positive characteristics and the negative characteristics, and outputting the probability that the characteristics belong to each category;
3) inputting the tongue image of the test sample and the blood tumor marker index into the trained model, and outputting the probability that the test sample is positive.

5. The system of claim 4, wherein:
the step of extracting the positive and negative features in step 1) comprises:
a coder extracts a characteristic vector of a tongue picture image, carries out splicing with a blood tumor marker index, carries out fusion through MLP of a fusion area, and outputs a positive characteristic f₁ and a negative characteristic f₂ after fusion;
simultaneously inputting f₁, f₂ and the spliced feature fₘ into the MLP of the feature selection area, and correspondingly outputting two control vectors g₁ and g₂, which respectively correspond to f₁ and f₂;
g₁ activates f₁ and f₂ respectively to form the selected features f₁⁺ and f₂⁻, g₂ activates f₁ and f₂ respectively to form the selected features f₁⁻ and f₂⁺, Two positive features f₁⁺ and f₁⁻ and two negative features f₂⁺ and f₂⁻ are obtained.

6. The system according to claim 1, wherein the discriminable features are obtained by dividing a tongue image into n small blocks and forming an input vector with a blood tumor marker index, and performing feature extraction to obtain deep features facilitating classification.

7. The system of claim 6, wherein the data processing module is configured to obtain a probability that the test sample is positive by:
a tongue picture image of a test sample is cut into small blocks to form an input sequence, a blood tumor mark index is placed at that end of the input sequence to form an input vector, a position index is added to the input vector, the input vector is led into a trained deep learn model to carry out feature extraction and feature fusion, selected deep features beneficial to classification are output, and the probability of belonging to each category is obtained.

8. The system according to claim 7, wherein the deep learning model is trained by the following steps:
a) cutting a tongue surface image into n small blocks, forming an input sequence according to the sequence, then placing a blood tumor marker index at the end of the input sequence to form an input sequence with the length of n+1, forming an input vector through linear mapping, and adding position indexes 0, 1, 2, . . . , n−1;
b) carrying out dimension amplification on the input blood tumor marker index through a full connection layer, aligning the input tumor marker index with an input vector mapped by a tongue surface image block, and endowing a position index n;
c) performing feature extraction and feature fusion by using an encoder based on the Transformer model, outputting the selected deep features which are beneficial to classification, and finally outputting the probability distribution of each category to which the deep features belong through the softmax classifier.

9. A tumor prediction method using the tumor prediction system based on tongue image and blood tumor markers according to claim 1, comprising:
tongue images and blood tumor marker indexes of the test sample are obtained;
inputting the tongue image and the blood tumor marker indicators of the test sample into the system to obtain the probability of tumor positivity for the test sample.

10. An application of the method accord to claim 9, comprising:
applying the method to predict tumors on a test sample.
